(19) United States
(12) Patent Application Publication
(10) Pub. No.: US 2019/0343772 A1
(43) Pub. Date: Nov. 14, 2019

(54) NANOFIBROUS MAT CONTAINING CERAMIC PARTICLES WITH RELEASABLE DOPANT

(71) Applicants: Nanopharma a.s., Pardubice (CZ); SG Ventures Pty Limited, Sydney (AU)

(72) Inventors: Christophe Jean Alexandre Barbé, Five Dock (AU); Aparajita Khatri, Sydney (AU); James Alexander Somerville, Alexanderia (AU); Liliana Berezkinova, Praha (CZ); Katerina Vodsedalkova, Kosmonosy (CZ)

(21) Appl. No.: 16/091,379

(22) PCT Filed: Apr. 5, 2017
(86) PCT No.: PCT/AU2017/050289

§ 371 Date: Oct. 4, 2018

(30) Foreign Application Priority Data
Apr. 5, 2016 (AU) 2016901261

(51) Int. Cl. A61K 9/70; D01F 1/10; D01F 6/34; D04H 1/728; D01D 5/00; A61K 31/167; A61K 47/32; A61K 9/51

(52) U.S. Cl. CPC A61K 9/703; D01F 1/10; D01F 6/34; D04H 1/728; D10B 2509/022; A61K 31/167; A61K 47/32; A61K 9/5115; D01D 5/0007

(57) ABSTRACT

A nanofibrous mat comprising: electrospun nanofibres forming said mat; and ceramic particles dispersed throughout said nanofibres and comprising a ceramic matrix and a dopant releasably encapsulated within said ceramic matrix, wherein the ceramic particles are dispersed throughout the nanofibres during electrospinning of the nanofibres, whereby said dopant is protected by said ceramic matrix during said electrospinning.

### CROSS-REFERENCE TO RELATED APPLICATION

This application is a U.S. National Stage of International Patent Application No. PCT/AU2017/050289, filed 5 Apr. 2017, which claims priority to Australian Patent Application No: 2016901261, filed on 5 Apr. 2016, the disclosure of which is incorporated herein by reference in its entirety.

### FIELD OF INVENTION

The present invention relates to a nanofibrous mat containing ceramic particles. The ceramic particles releasably encapsulate a dopant that is to be delivered from the nanofibrous mat. In particular, the invention relates to nanofibrous mats that contain at least one dopant encapsulated in a ceramic matrix. The invention also provides nanofibrous mats that contain at least one dopant encapsulated in a ceramic matrix and at least one free dopant, which may be the same as or different to the encapsulated dopant. Methods for the production of the nanofibrous mats are also considered.

### BACKGROUND ART

Nanofibres, generally considered fibres with diameters of less than 1 μm, have the potential to improve many products for numerous applications. They offer unique physical, mechanical, and electrical properties associated with their very high surface area. In that regard, nanofibre-nonwoven mats generally have very small pore sizes compared with commercial textiles.

Nanofibres are generally suitable for use in the production of nonwoven mat for controlled drug delivery. Electrospun fibres have desirable properties, such as high loading, simultaneous delivery of diverse therapies, ease of operation, and cost-effectiveness, which have expanded their use in drug delivery. Of the various applications, wound-dressing and local cancer treatments are two of the most investigated areas.

Electrospinning is a technology for the production of nanofibres that employs electrostatic forces to produce ultra-fine fibres with diameters ranging from micrometres down to hundreds of nanometres. This is currently quite well known technology for the production of ultra-fine fibres through the action of an external and internal electric field. The electrospinning set-up is composed of a spinning electrode (spinneret), which is connected to a high voltage source. The spinneret is usually positively charged and located at a defined distance from the oppositely charged collector. Different electrospinning set-ups can be used, including a horizontal set-up or vertical set-up with the spinneret located above and under the collector respectively. Different kinds of collectors can be used for electrospinning depending on the desired structure of the nanofibres. There are static or rotating collectors with a smooth or structured surface. A polymer solution is fed to the spinneret and a polymeric droplet is created in an orifice of this spinneret. The polymer liquid is drawn and elongated by electric forces and collected on the grounded collector as nanofibres.

Electrospun nanofibers have been successfully used to achieve different controlled drug release profiles, such as immediate, smooth, pulsatile, delayed, and biphasic releases. Drugs can be embedded in the fibre through dissolution or dispersion in the polymer solution. Many interesting biological entities for tissue development, for example proteins or nucleic acid in nature, do not dissolve in organic solvent and may suffer loss of bioactivity when dispersed in the polymer solution.

Commercial wound dressings that exert their antimicrobial effect by eluting germicidal compounds have been developed to provide sustained release of therapeutic doses of silver ions to the wound. However, silver ions are highly toxic to keratinocytes and fibroblasts, and may delay wound repair if applied indiscriminately to healing tissue areas. In addition to this, constant wound cleaning and redressing is needed, causing discomfort to patients and requiring substantial nursing input. A biodegradable drug-eluting wound dressing from an electrospun nanofibrous matrix potentially offers several advantages over conventional ones.

Depending on the wound type and its healing, the most suitable wound dressing system must be used. For rapid wound healing, it is common for different types of wound dressing materials to be used. Because of the unique properties of nanofibre structures the applications of these materials on various types of wounds is more attractive when compared to other modern wound dressing materials, such as hydrocolloids, hydrogels, and so forth. Generally, it may be considered that all modern wound dressing materials should maintain a suitable environment at the wound/dressing interface, absorb excess exudates without leakage to the surface of a dressing, provide thermal insulation, provide mechanical and bacterial protection, allow gaseous and fluid exchanges, absorb wound odour, be non-adherent to the wound and easily removed without trauma, provide some debridement action (remove dead tissue and foreign particles), be non-toxic, non-allergic and non-sensitising (to both patient and medical staff), and be sterile and non-scaring.

Electrospun drug-loaded nanofibre membranes potentially offer several advantages. Local antibiotics and anaesthetic have the advantage of delivering high drug concentrations to the precise area required, and the total dose of antibiotic applied locally is not normally high enough to produce toxic systemic effects. Antibiotic-loaded wound dressings made out of biodegradable polymeric membranes boast a number of further advantages. Firstly, biodegradable membranes provide bactericidal concentrations of antibiotics for the prolonged time needed to completely treat the particular infection. Secondly, versatility with regards to degree of biodegradability from weeks to months may allow many types of infections to be treated. Thirdly, biodegradable membranes dissolve, thus there is no need for removal. As these membranes dissolve slowly, they could potentially provide an in situ scaffold for wounds requiring regeneration of tissues, e.g. the soft tissue or bone defect will slowly fill up with tissue, minimising the need for reconstruction.

Topical application of drugs with an analgesic effect locally to skin or to surrounding tissues is often used in different pain settings, usually with the aim of blocking or reducing activation of nociceptive nerve endings and propagation of action potential to the central nervous system. One of the most widely used class of drugs for this purpose is local anaesthetics, which work as voltage gated sodium channel blockers. They are used for surface anaesthesia, where a spray, solution or cream is applied to the skin or to a mucous membrane and the effect is usually short-lived and limited to the area of contact. Another method involves infiltration anaesthesia, where local anaesthetic is injected and/or infused into the tissue to be anesthetized. Peripheral nerve block is used to anaesthetize the area innervated by a peripheral nerve, in which an injection of a local anaesthetic is made in the vicinity of the affected area.

For topical application, local anaesthetics are currently available in the form of solution, cream and patch. The solution is used for injections, infiltration and as a spray, while the duration of the analgesic/anaesthetic effect is usually defined by the pharmacological profile of the local anaesthetic used. Cream formulations are used directly on the skin, but are difficult to maintain for a longer time period.

The Lidocaine patch represents a relatively new possibility for local anaesthetic delivery and is used in a number of different clinical settings besides treatment of neuropathic pain. A number of new local anaesthetic formulations are now being developed for extended effect and reduced systemic toxicity, using liposomes, polymers and microspheres (Weiniger, C. F., L. Golovanevski, A. J. Domb and D. Ickowicz. Extended release formulations for local anaesthetic agents. Anaesthesia. 2012, 67(8), 906-916.). A nanofibre based system has also been developed and used for wound dressing (Chen, Dave W., Yung-Heng Hsu, Jun-Yi Liao, Shih-Jung Liu, Jan-Kan Chen and Steve Wen-Neng Ueng. Sustainable release of vancomycin, gentamicin and lidocaine from novel electrospun sandwich-structured PLGA/collagen nanofibrous membranes. International Journal of Pharmaceutics. 2012, 430, 335-341) and for epidural analgesia, where lidocaine-embedded poly([D,L]-lactide-co-glycolide) nanofibres reduced the severity of pain in rats after laminectomies (Tseng, Y Y, W A Chen, J Y Liao, Y C Kao And S J Liu. Biodegradable poly([D,L]-lactide-co-glycolide) nanofibers for the sustainable delivery of Lidocaine into the epidural space after laminectomy. Nanomedicine. 2014, 9, 77-87). In this study, the nanofibres provided a sustained release of lidocaine for more than 2 weeks, and the local concentration was much higher than the concentration in plasma.

The subject matter claimed herein is not limited to embodiments that solve any disadvantages or that operate only in environments such as those described above. Rather, this background is only provided to illustrate one exemplary technology area where some embodiments described herein may be practice.

### SUMMARY OF INVENTION

As mentioned above, the present invention relates generally to nanofibrous mats that contain at least one dopant encapsulated in a ceramic matrix, and possibly at least one free dopant, which may be the same as or different to the encapsulated dopant. Methods for the production of the nanofibrous mats are also described.

According to one aspect of the invention there is provided a nanofibrous mat comprising:
electrospun nanofibres forming said mat; and
ceramic particles dispersed throughout said nanofibres and comprising a ceramic matrix and a dopant releasably encapsulated within said ceramic matrix,
wherein the ceramic particles are dispersed throughout the nanofibres during electrospinning of the nanofibres, whereby said dopant is protected by said ceramic matrix during said electrospinning.

It has been found that a dopant may be incorporated into a nanofibrous mat during electrospinning by protecting the dopant within a ceramic matrix that releasably encapsulates the dopant. When the nanofibrous mat has been formed, the encapsulated dopant may be selectively released from the ceramic matrix to achieve its purpose.

The electrospun nanofibres may be formed from any material suitable for use in electrospinning. The electrospun nanofibres may include biodegradable polymers and non-biodegredable polymers. Importantly, though not necessarily essentially, the dopant encapsulated inside the particles is poorly soluble in the solvent of the polymeric solution to be electrospun (i.e. water or alcohol or another organic solvent). If not, the content of the particles may be prematurely released in the polymer solution and thus incorporated in the fibres as “free drug”. It may be acceptable for the particles to be slightly soluble, but preferably not highly soluble.

In certain embodiments, the electrospun nanofibres are selected from the group consisting of biocompatible and biodegradable or non-biodegradable synthetic or natural polymers. For example, the electrospun nanofibres may be selected from the group consisting of cellulose acetate, collagen, elastin, gelatin, hyaluronic acid, polyacrylonitrile, polycaprolactone, polydioxanone, polyethylene oxide, polyhydroxybutyrate, poly(D-lactide), poly(D,L-lactide-co-caprolactone), poly(D,L-lactide-co-glycolide) (PLGA), polylactide, poly(L-lactide), poly(L-lactide-co-caprolactone-co-glycolide), polypropylene, polytetrafluorethylene, polyvinylpyrolidone, sodium alginate and zein. In a preferred embodiment, the electrospun nanofibres are formed from polyvinylalcohol (PVA). For example, a 12 wt. % PVA solution is preferred.

The ceramic particles dispersed throughout the nanofibres comprise a dopant releasably held within a ceramic matrix. The particles may comprise solid, porous spheres, or may take the form of a core with one or more layers surrounding the core. If the latter, the dopant may be located in the core, shell or both. The same or different dopants may be included in the core and shell.

The ceramic matrix may be a polymerisation and/or condensation and/or crosslinking product of a precursor material. It may be a hydrolysed silane, such as a hydrolysed organosilane. It may comprise an organically modified ceramic, such as an organically modified silica (organo-silica). It may be a ceramic having bound organic groups. The bound organic groups may be ethyl, propyl, isopropyl, butyl, isobutyl, tert-butyl, pentyl, hexyl, isooctyl, decyl, dodecyl, cyclohexyl, cylcooctyl or cyclopentyl. These may be substituted (e.g. with functional groups, halogens, aryl groups, etc.) or may be unsubstituted. Other suitable organic groups include aryl groups, which may have between about 6 and 14 carbon atoms, and may have for example, 6, 8, 10, 12 or 14 or more than 14 carbon atoms. Examples include phenyl, biphenyl, naphthyl and anthracyl. These may each, optionally, be substituted by one or more alkyl groups (e.g. C1 to C6 straight chain or branched alkyl), halogens, functional groups or other substituents. The organic group may be an alkenyl or alkynyl or benzyl group. The alkenyl or alkynyl group may have between 2 and about 18 carbon atoms, and may be straight chain, branched or (if sufficient carbon atoms are present) cyclic. It may have 1 or more than 1 double bond, or 1 or more than 1 triple bond, and may have a mixture of double and triple bonds. If the group has more than one unsaturated group, the unsaturated groups may be conjugated or unconjugated. The solid matrix may comprise chemical groups derived from a catalyst used in the formation of the ceramic particles, and the groups may be on the surface of the particles. If a surfactant used in the formation reaction is capable of combining chemically with the precursor material, the matrix may comprise chemical groups derived from the surfactant. For example, if the precursor material comprises an organotrialkoxysilane, and the catalyst comprises a trialkoxyaminoalkylsilane, then the matrix may comprise aminoalkylsilyl units. These may be distributed evenly or unevenly through the particle. They may be preferentially near the surface of the particle. They may provide some degree of hydrophilicity, e.g. due to amino functionality, to the particle surface. Additionally, the surfactant may be capable of combining chemically with the precursor material. For example if the precursor material comprises an organotrialkoxysilane, and the surfactant comprises trialkoxysilyl functionality, then the matrix may comprise surfactant derived units. The surfactant may be adsorbed on the surface of the particle.

The dopant may be selected from the group consisting of hydrophobic and hydrophilic small molecule drugs such as antibiotics (Chloremphenicol), analgesics (nonsteroidal anti-inflammatory drugs (e.g. diclofenac and ibuprofen), dibucaine, bupivacaine, capsaicin, amitriptyline, glyceryl trinitrate, opioids, menthol, pimecrolimus, and phenytoin), Scopolamine (tropane alkaloid drug) for motion sickness. It may include proteins for therapeutic purposes: Steroid hormones (skin eczema or birth control, HRT, estrogen or testosterone), growth factors, cytokines, antibodies (for wound healing), vaccines (buccal patch), Nitroglycerine for Angine (sublingual patch), Vitamin B12. The dopant may be a fluorescent or radioactive or a metal (e.g. gold) tracer to study a biological process or monitor or diagnose a condition. In one particular embodiment, the dopant is Lidocaine.

The dopant, which may be a hydrophobic material, a hydrophilic material, an oligo (DNA & RNA), or a protein, etc., may represent between about 0.01 and 50% of the weight or the volume of the particle, or between about 0.01 and 10%, 0.01 and 1%, 0.01 and 0.5%, 0.01 and 0.1%, 0.01 and 0.05%, 0.1 and 30%, 1 and 30%, 5 and 30%, 10 and 30%, 0.1 and 10%, 0.1 and 1% or 1 and 10% of the weight or the volume of the particle, and may represent about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25 or 30% of the weight or the volume of the particle.

The diameter of the particles may be somewhat dictated, although not necessarily, by the size of the electrospun nanofibres. For example, the diameter of the particles should be such that the particles may be incorporated in the nanofibres of the mat without compromising the integrity of the fibres of the mat. For example, the particle may have a diameter between about 1 nm and about 1000 nm. Although one would think that the particles should be smaller than the fibres, this is not imperative. It has been found that aggregates which are larger than the fibres can be incorporated inside the fibres given a swollen aspect of the fibres. Generally, the particle diameter is preferably <1.5 times the diameter of the fibres, more preferably smaller than the diameter of the fibres, and even more preferably ½ of the fibre diameter.

The particles may be spherical, oblate spherical or may be ovoid or ellipsoid. They may be regular or irregular shaped. They may non-porous, or may be mesoporous or microporous. It may have a specific surface area of between about 2 and 400 m²/g, or between about 2 and 25, 2 and 20, 2 and 15, 2 and 10, 10 and 50, 10 and 25, 15 and 25 or 20 and 50 m²/g, and may have a specific surface area of about 2, 3, 4, 5, 6, 7, 8, 9, 10, 11, 12, 13, 14, 15, 16, 17, 18, 19, 20, 21, 22, 23, 24, 25, 26, 28, 30, 35, 40, 45 or 50 m²/g.

The dopant is capable of being released from the particle, for example over a period of time. The release may be at a controlled or sustained rate. The particles may be capable of releasing the dopant over a period of between about 1 minute and 2 weeks. The rate of release of the dopant may be characterised by a half-release time, which is the time after which half of the original amount of hydrophobic material has been released. The particle(s) may have a half-release time of between about 1 minute and 96 hours. The particles may therefore be used in applications requiring sustained release over relatively short periods, for example between about 1 minute and about 1 hour, or they may be used in applications requiring sustained release over intermediate periods, for example between about 1 hour and about 1 day, or they may be used in applications requiring sustained release over relatively long periods, e.g. greater than 1 day.

The particles may be in the form of a composition together with an acceptable carrier, diluent, excipient and/or adjuvant. Where the dopant is a pharmaceutical substance the carrier may be a pharmaceutically acceptable carrier and the particles may be pharmaceutically acceptable, where the dopant is a veterinary substance the carrier may be a veterinarilly acceptable carrier and the particles may be veterinarilly acceptable, where the dopant is a biocidal substance the carrier may be a biocidally acceptable carrier and the particles may be biocidally acceptable, where the dopant is a cosmetic substance the carrier may be a cosmetically acceptable carrier and the particles may be cosmetically acceptable, and where the dopant is a fungicidal substance the carrier may be a fungicidally acceptable carrier and the particles may be fungicidally acceptable.

The encapsulation of dopant is achieved substantially in accordance with processes disclosed in International Publication No. WO 2006/133519, the content of which is incorporated herein in its entirety. The encapsulation may be achieved in accordance with the processes disclosed in International Publication Nos. WO 2001/062232 WO 2006/050579, WO 2006/084339 and WO 2012/021922, which are also incorporated in their entirety.

It has been surprisingly found that efficacy may be dramatically increased by including in the nanofibrous mat a combination of ceramic particles comprising a ceramic matrix and a dopant releasably encapsulated within said ceramic matrix and free dopant. As used herein “free dopant” is intended to mean dopant that is not encapsulated in a ceramic matrix, for example in powder form. As such, in other embodiments the nanofibrous mat comprises additionally comprises free dopant dispersed throughout the nanofibres. The free dopant may be the same as or different to the encapsulated dopant. Generally, the free dopant is the same as the encapsulated dopant.

Accordingly, in another aspect the invention provides a nanofibrous mat comprising:
electrospun nanofibres forming said mat;
ceramic particles dispersed throughout said nanofibres and comprising a ceramic matrix and a dopant releasably encapsulated within said ceramic matrix; and
free dopant dispersed throughout the nanofibres.

The abovementioned options are equally applicable to this aspect of the invention and are explicitly incorporated herein by reference.

In a further aspect of the invention there is provided a method of forming a nanofibrous mat comprising:
providing an electrospinning solution containing a precursor to electrospun nanofibres;
adding ceramic particles comprising a ceramic matrix and a dopant releasably encapsulated within said ceramic matrix to said electrospinning solution; and
electrospinning the electrospinning solution comprising said ceramic particles to form said nanofibrous mat having said ceramic particles dispersed throughout the formed electrospun nanofibers.

As noted above, it has been found that surprising results may be obtained is free dopant is also distributed throughout the nanofibrous mat. As such, it is preferred that the method comprises adding dopant in powder form to the electrospinning solution; and electrospinning the electrospinning solution comprising the ceramic particles and dopant in powder form to form the nanofibrous mat having the ceramic particles and dopant dispersed throughout the formed electrospun nanofibers. This may advantageously increase the amount of dopant in the resulting layer.

The electrospinning conditions may be selected generally depending on the nanofibres selected. Exemplary ranges for electrospinning parameters are provided in Table 1 below.

TABLE 1
Parameters for electrospinning
Minimum-maximum ranges

| | |
|---|---|
| Distance of electrodes [mm] | 120-180 |
| Voltage [kV] | 10-60 |
| Flow rate [ml/h] | 1-20 |
| Unwinding speed [mm/s] | 0.1-1 |
| Temperature [° C.] | 20-30 |
| RH [%] | 30-50 |

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

The present invention consists of features and a combination of parts hereinafter fully described and illustrated in the accompanying drawings, it being understood that various changes in the details may be made without departing from the scope of the invention or sacrificing any of the advantages of the present invention.

### BRIEF DESCRIPTION OF ACCOMPANYING DRAWINGS

To further clarify various aspects of some embodiments of the present invention, a more particular description of the invention will be rendered with references to accompanying drawings in which:

FIG. 1 illustrates a flow diagram outlining a process for encapsulation of Lidocaine.

FIG. 2 illustrates a graph of TGA/DTA analysis of particles containing Lidocaine.

FIG. 3 illustrates a graph of Static light scattering analysis of particle containing Lidocaine.

FIG. 4 illustrates a SEM image of particles containing Lidocaine.

FIG. 5 illustrates a TEM image of particles containing Lidocaine.

FIG. 6 illustrates the release profile of free and ceramic-encapsulated Lidocaine from fibres in the Sotax USP 4 system.

FIG. 7 illustrates a Franz cell apparatus.

FIG. 8 illustrates SEM images of nanofibres: A) Nanofibres with Lidocaine powder (5 000×), B) Nanofibres with Lidocaine powder (25 000×), C) Nanofibres with Lidocaine in spheres (5 000×), D) Nanofibres with Lidocaine in spheres (25 000×), E) Nanofibres with Lidocaine powder and Lidocaine in spheres (5 000×), F) Nanofibres with Lidocaine powder and Lidocaine in spheres (25 000×), G) PVA Nanofibres (5 000×), H) PVA Nanofibres (25 000×).

FIG. 9 illustrates the permeation profile of Lidocaine through the human skin over 48 H-Comparison between nanofibre patches with free and/or ceramic encapsulated Lidocaine and the commercial patch.

FIG. 10 illustrates a graph of quantity of Lidocaine released from the patches through the skin.

FIG. 11 illustrates a graph of quantity of Lidocaine released from the patches in the dermis.

FIG. 12 illustrates a graph of quantity of Lidocaine released from the patch in the epidermis.

FIG. 13 illustrates a graph of quantity of remaining Lidocaine in the patch-comparison between nanofibre patches.

FIG. 14 illustrates a graph of quantity of remaining Lidocaine in the patch-comparison between nanofibre patches and the commercial patch.

FIG. 15 illustrates the permeation profile of free Lidocaine in water through human skin.

FIG. 16 illustrates the release of Lidocaine (through the skin) at times earlier than 12 hours.

FIG. 17 illustrates Lidocaine released through the skin at different times for up to 48 hours.

FIGS. 18-19 illustrate Lidocaine released from the new batch of Lidocaine nanofibre mats over 24 hours in two separate experiments.

FIG. 20 illustrates a comparison of the combination patch and the commercial patch as percentage (%) of Lidocaine permeated at 24 H through the human skin averaged over 4 separate studies.

FIG. 21 illustrates the penetration of 60 nm particles embedded in nanofibre mat in different layers of stratum corneum from human skin: the graph shows concentration of nanoparticles (calibrated to fluorescence levels) in various layers of stratum corneum over 24 hours.

### EXAMPLES

Material for the Production of Encapsulated Lidocaine

Lidocaine and Tetrahydrofuran (THF), Phenyltrimethoxy silane (PTMS) and (3-Aminopropyl)triethoxysilane (APTES), Tetraethylorthosilicate (TEOS) and Tertigol NP-15 (NP-15).

Encapsulated Lidocaine Production

The encapsulation of Lidocaine was achieved substantially in accordance with processes disclosed in International Publication No. WO 2006/133519, the disclosure of which is incorporated herein in its entirety, as discussed above.

Typically, this process yields particles within a diameter range of 200-1000 nm, which is too large for incorporation efficiently in the nanofibres (Diameter range: ˜100-300 nm). Therefore, the process was developed further to allow for the incorporation of particles into the fibres. This involved reduction in the average size of the particles through adjusting the reagent ratios and manipulation of the emulsion properties by changing to a surfactant with a higher hydrophile-lipophile balance than has been typically used.

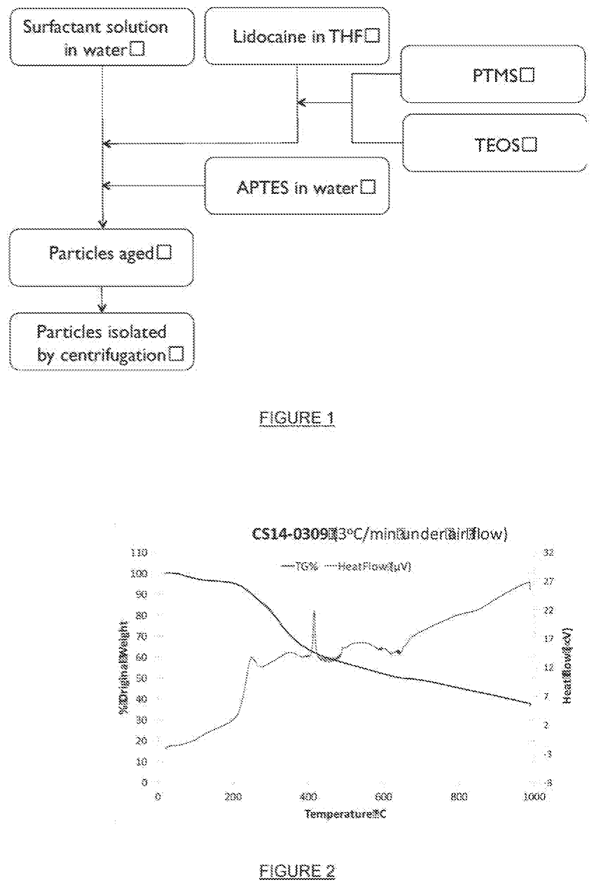

Specifically for Lidocaine encapsulation, the outline of the modified process is illustrated in the flow chart of FIG. 1. A surfactant solution was prepared by dissolving 54 g of NP-15 in 400 mL of water. The APTES was hydrolysed by mixing 22.4 mL of APTES with 22.4 mL of water and allowed to cool. 12.8 g of Lidocaine was dissolved in 6.4 mL of THF, followed by the addition of 22.09 g of PTMS and 13.44 of TEOS. The Lidocaine/PTMS/TEOS mixture was added to the surfactant solution and allowed to fully mix. 60 minutes after the addition of the Lidocaine solution the hydrolysed APTES solution was added. The particles were aged overnight before being separated by centrifugation (10 minutes at 12,000 rpm) and collected for analysis and further experimentation.

Analysis of Particles

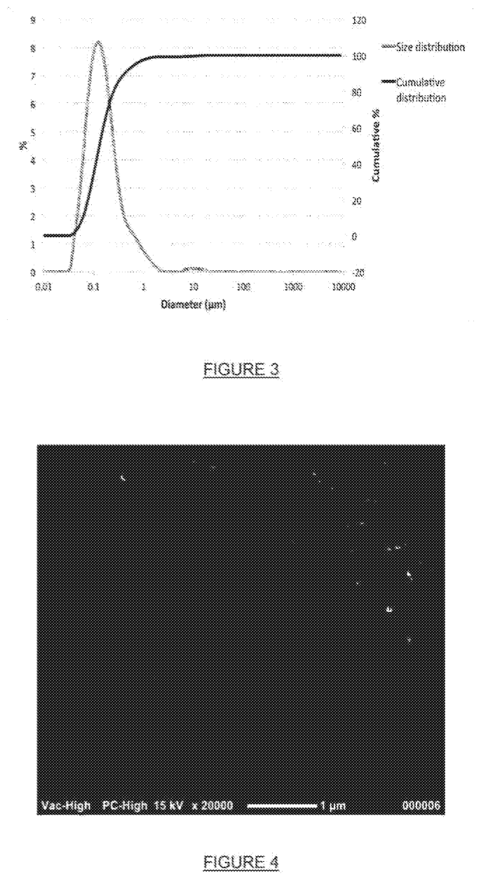

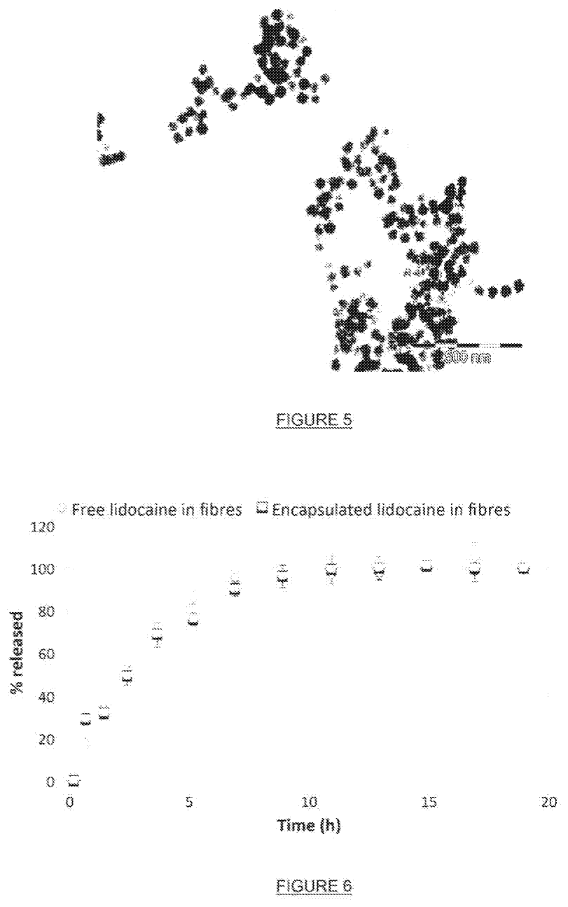

Following production, particles were characterised by a range of techniques to confirm the composition, the size and the loading of the particles. The composition was determined by thermal gravimetric analysis/differential thermal analysis (TGA/DTA) and was consistent with previous particle formulations (FIG. 2). The size was determined by static light scattering (Malvern Mastersizer 2000 μu) (FIG. 3), scanning electron microscopy (SEM, Jeol NeoScope JCM-5000) (FIG. 4) and transmission electron microscopy (TEM, Philips CM10) (FIG. 5). These analyses showed that spherical particles of ˜60 nm were produced showing a uniform morphology.

The loading of the particles was determined using High Performance Liquid Chromatography (HPLC). For this, Lidocaine was leached form the particles using ethanol and the particles were subsequently removed from the solution by centrifugation. The supernatant was then analysed by HPLC to determine the loading. In the case of Lidocaine particles, the loading ranged from 10-15 wt

Material for the Production of Nanofibers

Polyvinylalcohol (PVA) is a synthetic hydrophilic polymer, which belongs to the group of vinyl polymers. PVA constitutes a simple chemical structure, which contains a functional hydroxyl group. It is soluble in polar solvents such as water. PVA is prepared by polymer-analogous hydrolysis or alcoholysis of polyvinyl acetate in methanol, when ester bonds are formed. Mowiol® 18-88 from Sigma Aldrich was used for the preparation of the polyvinyl alcohol solution.

Electrospinning

PVA was dissolved in distilled water at an elevated temperature of 60° C. with constant stirring for 24 hours to achieve a concentration of 12 wt %. Various amounts of Lidocaine in three forms (powder, encapsulated and a combination of both) were added into the PVA solutions. A QSonica sonicator was used for homogeneous dispersion of the Lidocaine in the PVA solutions. All solutions containing Lidocaine or Lidocain ceramic particles were electrospun using an electrospinning device. Nanofibers were collected on nonwowen textile. A needle with a 1.6 mm diameter was used as the electrode.

The aim was to optimize production to determine the maximum amount of lidocaine in the polymer solution that can be productively and effectively electrospun. The nanofibrous layers with the highest possible amounts of Lidocaine were made for tests using a Franz diffusion cell.

Electrospinning Conditions

All parameters of the electrospinning process are described in the following tables.

TABLE 2
Production of nanofibres with Lidocaine powder
Solution of 10 g 12 wt % PVA with 0.6 g Lidocaine

| | |
|---|---|
| Concentration of Lidocaine [%] | 5.66 |
| Distance of electrodes [mm] | 150 |
| Voltage [kV] | −10; +20 |
| Flow rate [ml/h] | 6 |
| Unwinding speed [mm/s] | 0.3 |
| Temperature [° C.] | 22 |
| RH [%] | 33 |
| Area weight [g/m²] | 20.6 |

TABLE 3
Production of nanofibres with Lidocaine in spheres
Solutions of 10 g 12 wt % PVA with 7.5 g of paste with spheres

| | |
|---|---|
| Concentration of spheres [%] | 42.86 |
| Amount of Lidocaine [g] | 0.31 |
| Distance of electrodes [mm] | 150 |
| Voltage [kV] | −10; +20 |
| Flow rate [ml/h] | 13 |
| Unwinding speed [mm/s] | 0.3 |
| Temperature [° C.] | 21 |
| RH [%] | 33 |
| Area weight [g/m²] | 20.6 |

TABLE 4
Production of nanofibres with combination
of Lidocaine powder and Lidocaine in spheres
10 g 12% PVA + 3 g paste with spheres + 0.5 g Lidocaine

| | |
|---|---|
| Concentration of spheres [%] | 0.126 |
| Amount of Lidocaine [g] | 3.89 |
| Distance of electrodes [mm] | 150 |
| Voltage [kV] | −10; +20 |
| Flow rate [ml/h] | 9 |
| Unwinding speed [mm/s] | 0.3 |
| Temperature [° C.] | 22 |
| RH [%] | 32 |
| Area weight [g/m²] | 20.6 |

TABLE 5
Production of PVA nanofibres without Lidocaine
10 g 12% PVA

| | |
|---|---|
| Distance of electrodes [mm] | 150 |
| Voltage [kV] | −10; +20 |
| Flow rate [ml/h] | 5 |
| Unwinding speed [mm/s] | 0.3 |
| Temperature [° C.] | 23 |
| RH [%] | 33 |
| Area weight [g/m²] | 20.6 |

HPLC Method

HPLC was used for quantitative determination of the amount of released Lidocaine from nanofibres. Typically, HPLC separation is based on the separation of analytes according to their distribution between stationary (chromatographic column) and mobile phases (liquid). In the case of Lidocaine, extracts from patches, from skin or the buffer were loaded onto a C18 reverse phase column and eluted in a mixture of acetonitrile and a 0.1% triflouroacetic acid solution. A UV-Vis detector was used to quantify the amount of Lidocaine present.

USP 4 Method

Release of Lidocaine form the fibres and particles was initially investigated using a Sotax CE7 smart USP4 system with nanoparticle adapters in a closed configuration. The system flows recirculated DI water over dialysis tubing containing the sample continually for the designated time period and collects fractions of the supernatant at various time points. The collected fractions are then analysed by HPLC. Using this method to compare fibres with free Lidocaine (i.e. Lidocaine not in particles) and fibres with encapsulated Lidocaine (i.e. Lidocaine in particles) it was found that there was no significant difference in the release profiles of the two types of fibres (FIG. 6). Thus, particles incorporated in the nanofibrous mat were able to release Lidocaine as efficiently as from mat containing free Lidocaine in the fibres.

Franz Cell Method

Though the USP4 apparatus is effective for release studies, it is not possible to detect the release kinetics of Lidocaine transdermally by this method. The barrier (in this case the skin) plays a major role during transdermal transmission. Transdermal penetration can be tested in vitro or in vivo. The classic method for testing transdermal in vitro absorption is a static vertical diffusion cell, called Franz cell (FIG. 7). Experiments are generally performed on human or animal skin. Human skin is the best standard for developing products for human use.

Typically in the Franz cell, a skin membrane separates the donor (upper) cell part from the acceptor (the lower). The membrane lies on the bottom surface of the donor part. The test substance is placed in a suitable medium into the donor part. The acceptor is filled with an acceptor liquid (usually a buffer at pH 7.4). This part is continuously stirred and samples are periodically taken out and analysed. Instrumental analysis of the collected samples is usually performed by HPLC, radiography or scintigraphy, depending on the type of substance being investigated.

Here, the Franz cell method with HPLC analysis was used to study of the release kinetics of Lidocaine from nanofibre layers. The next section describes the production and optimization of nanofibre layers with Lidocaine powder, Lidocaine-spheres and a combination of Lidocaine powder and Lidocaine-spheres.

Characterization of Nanofibrous Layers

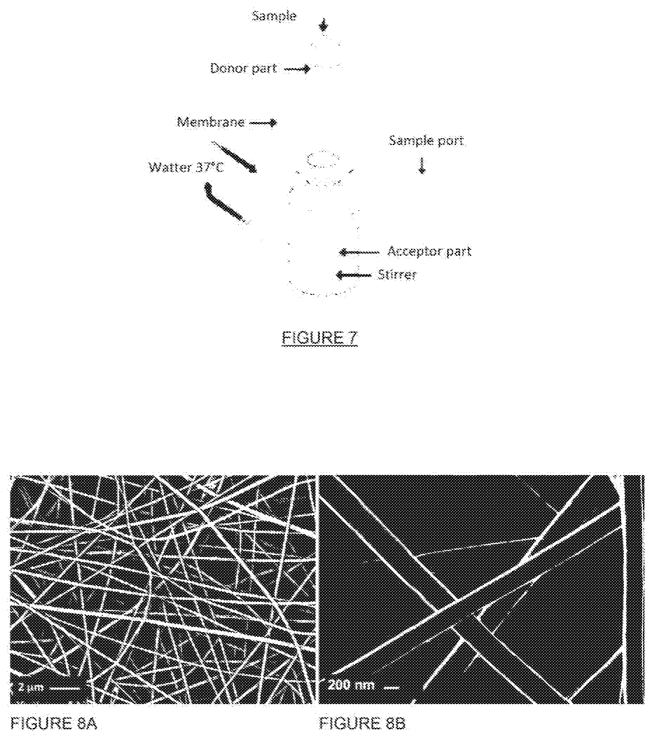

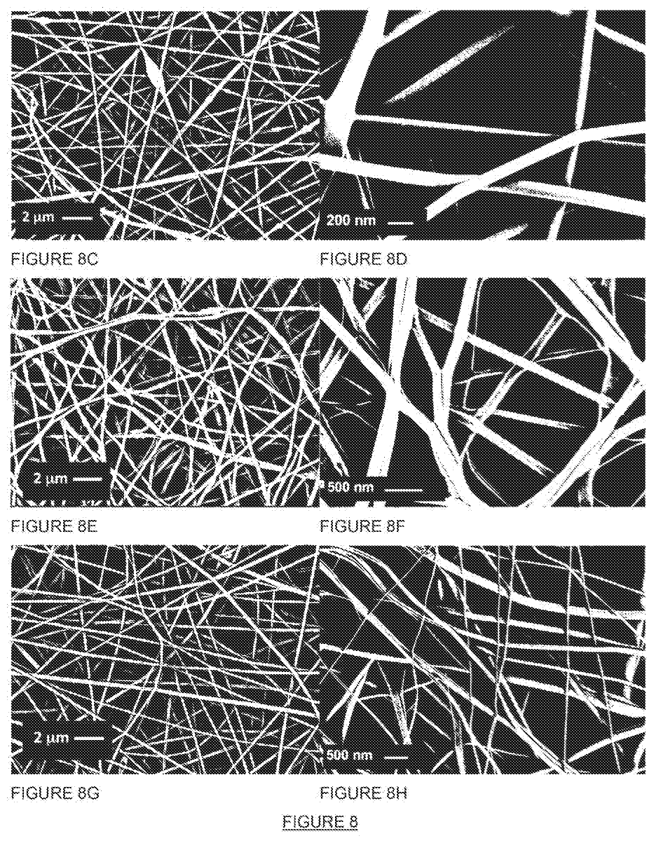

Nanofibrous structures were analysed using scanning electron microscope (SEM) (Zeiss), after sputter coating with gold. Diameters of the electrospun fibres were analysed from the SEM images using image analysis software (NIS Elements). Diameters of nanofibres were up to 300 nm for all prepared layers (see FIG. 8).

Comparison of Lidocaine Release from Different Types of Nanofibrous Mats Using Franz Cell Method

Permeation analysis of Lidocaine immobilised in various ways (i.e. directly in nanofibres, in ceramic particles in nanofibres, etc.) into and through the skin was performed using a Franz cell method. Human skin was used as the barrier membrane. Samples were subsequently analysed by HPLC. Phosphate buffered saline (PBS) with pH 7.4 was used as the acceptor buffer. It was continuously stirred at 32° C. Samples with an area of area 2 cm² were analysed for each nanofibre layer.

The aim of the experiment was to obtain permeation profiles of immobilized Lidocaine from the nanofibrous layers through the human skin. Lidocaine was immobilized into a nanofibre layer directly by electrospinning Lidocaine powder in PVA or encapsulated Lidocaine in PVA, or Lidocaine was immobilized by a combination of these methods. Characteristics of the samples are shown in Table 6. A commercial patch “Versatis” containing Lidocaine was analysed with these samples. Residues of Lidocaine in nanofibrous layers (donor of Lidocaine) and in the skin (epidermis and dermis) were analysed after the experiment. The experiment schedule is schematically shown in Table 7.

TABLE 6
Characteristics of analysed samples

| | | Lidocaine | Area weight |
|---|---|---|---|
| sample | Immobilization | (%) | of patch |
| 1 | Control - only nanofiber patch | 0.00% | 20.6 g/m² |
| 2 | nanofiber patch with in spheres | 1.77% | 20.6 g/m² |
| | encapsulated lidocaine | | |
| 3 | nanofiber patch with free lidocaine | 5.66% | 20.6 g/m² |
| 4 | combination of sample 2 and 3* | 4.62% | 20.6 g/m² |
| 5 | commercial patch with lidocaine** | 5.00% | 0.1 g/cm² |

*concentration of Lidocaine in spheres = 1.29%, concentration of free Lidocaine in buffer = 4.83%
**commercial patch “Versatis”

TABLE 7
Experimental results

| | PVA | Nanofibre | Nanofibre | | | |
|---|---|---|---|---|---|---|
| | nanofibre | patch with | patch with | Combination | Commercial | Number |
| | patch | Lidocaine in | free | of sample 2 | patch with | of |
| | (sample | ceramispheres | Lidocaine | and 3 | Lidocaine | analysed |
| donor: | 1) | (sample 2) | (sample 3) | (sample 4) | (sample 5) | samples |

time points (acceptor buffer)

| | | | | | |
|---|---|---|---|---|---|
| 0 | 4 | 9 | 9 | 9 | 9 |
| 12 h | 4 | 9 | 9 | 9 | 9 |
| 16 h | 4 | 9 | 9 | 9 | 9 |
| 20 h | 4 | 9 | 9 | 9 | 9 |
| 24 h | 4 | 9 | 9 | 9 | 9 |
| 36 h | 4 | 9 | 9 | 9 | 9 |
| 40 h | 4 | 9 | 9 | 9 | 9 |
| 44 h | 4 | 9 | 9 | 9 | 9 |
| 48 h | 4 | 9 | 9 | 9 | 9 |

terminal analysis

| | | | | | |
|---|---|---|---|---|---|
| Donor | 4 | 9 | 9 | 9 | 9 |
| epidermis | 4 | 9 | 9 | 9 | 9 |
| Dermis | 4 | 9 | 9 | 9 | 9 |

TABLE 8
Lag time of free Lidocaine (from the data of FIG. 9)

| Sample (5% lidocaine) | Calculated lag time (h) | Average of lag time (h) |
|---|---|---|
| 1 | 12.85 | 15.03 |
| 2 | 15.09 | |
| 3 | 13.09 | |
| 4 | 18.16 | |
| 5 | 15.96 | |

### DISCUSSION

The lag time of Lidocaine in an aqueous solution (FIG. 15) was found to be 15 hours (Table 8) in the skin. For this reason, in the first set of permeation studies, the samples for permeation analysis through the skin were collected after 12 hours.

Permeation profiles of nanofibrous layers showed faster penetration through the skin in the first 12 hours than Lidocaine in aqueous solution. It is considered that this rapid transmission of Lidocaine could be due to degradation of PVA to metabolites (acetate esters, pyruvate, lactate, etc.), which are then transported into cells through active transport or passive diffusion. It is possible that Lidocaine penetrates with these metabolites (e.g. as an active symporter) or that these metabolites act as penetration enhancers for Lidocaine.

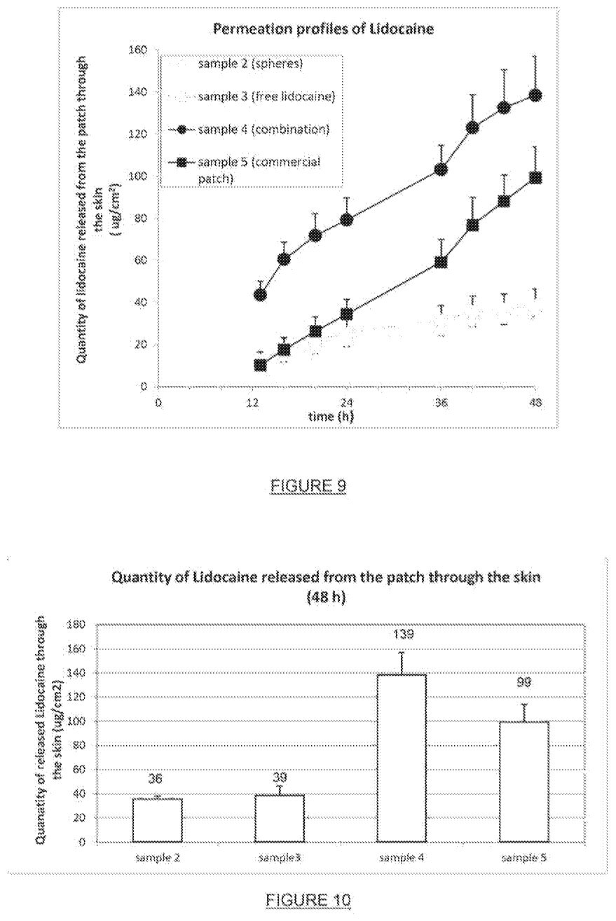

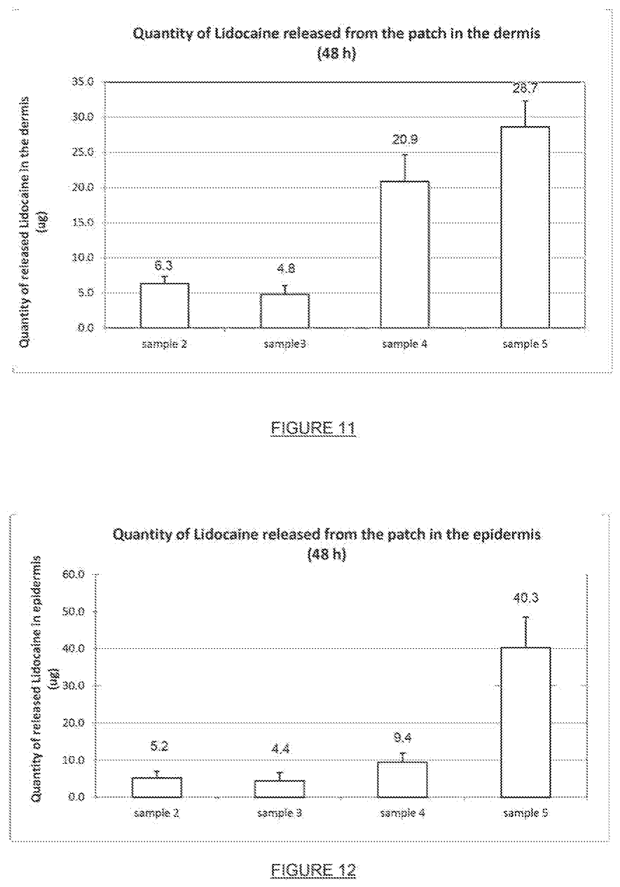

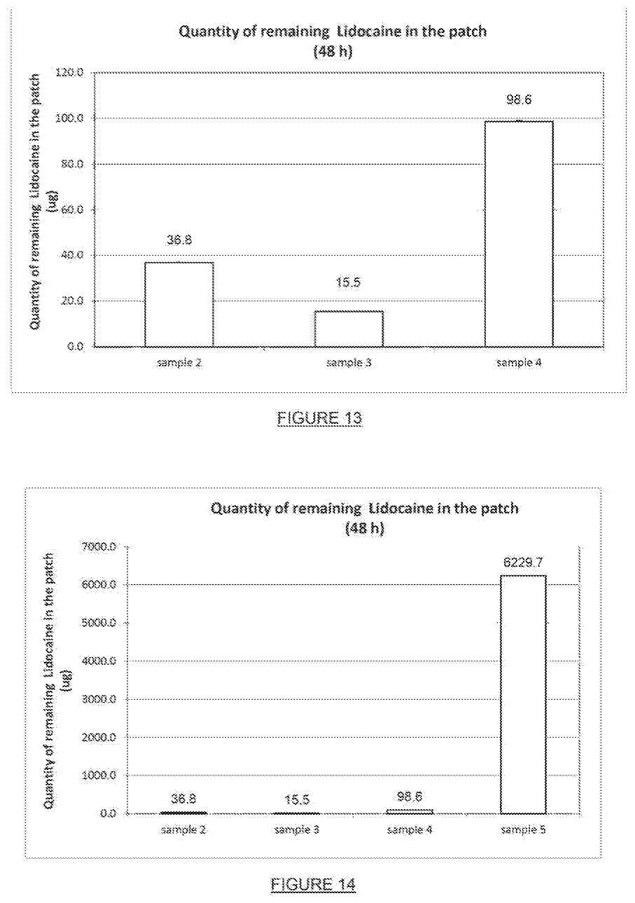

The permeation profile of a commercial patch Versatis (sample 5) showed a linear release (FIG. 9) during the experiment (48 hr). The amount of released Lidocaine in 12 hours was comparable to the amount of released Lidocaine from nanofibre samples 2 and 3 and about 4 fold lower than the combination patch (sample 4). In terms of percentage of Lidocaine permeation through the skin, the nanofibre mats were far superior to the commercial patch (70-85% vs. only 4.1% released from the commercial patch). The data from 48 hr terminal analysis of the donors (remainder Lidocaine in the patch) (FIG. 13 and FIG. 14) indicated that >95% of the Lidocaine was still remaining behind in the commercial patch while, in comparison, only 15%-30% was residual in nanofibre samples 2, 3 and 4. The accumulation of Lidocaine in the dermis and epidermis was higher for the commercial patch (˜69 μg) than for nanofibre layers (Range: 10-30 μg) (FIG. 12 and FIG. 13). The total amount of released Lidocaine through the skin at 48 hours showed highest release from the combination mat (130 μg/cm2) followed by commercial patch (99 μg/cm2) and then from nanofibre mat samples #2 and 3 (36 and 39 μg/cm2, respectively) (FIG. 10).

Interestingly, when release rates were normalised to initial Lidocaine loadings, sample 2 and sample 4 (the nanofibrous mats with encapsulated Lidocaine), showed the greatest efficiency of permeation at all times (20%-80%), with the commercial patch showing only 2% permeation at 48 hr.

Permeation profiles of nanofibrous layers with Lidocaine in spheres (sample 2) or Lidocaine powder (sample 3) were very similar, there were no significant differences between them (FIG. 9). For both, the majority of the Lidocaine was released after 24 hours and the following release of Lidocaine is linear. As initial amounts of Lidocaine loadings was 3 fold more in the mat with free Lidocaine, this indicated potential synergistic effect between the two technologies. Further, the amount of released Lidocaine from samples 2 and 3 was comparable to the amount of Lidocaine released from the commercial patch after 12 hours, indicating the superior penetration of Lidocaine when nanofibrous mats were used.

Immobilization of Lidocaine into a nanofibrous layer by a combination of encapsulated Lidocaine and Lidocaine powder (sample 4) showed a very different permeation profile. Samples 2, 3 and 5 showed a similar amount of released Lidocaine through the skin after 12 hours, however sample 4 exhibited up to 4 times higher released Lidocaine through the skin after 12 hours (FIG. 9). This again suggested a beneficial interaction between encapsulated Lidocaine and nanofibres facilitating better permeation profiles for Lidocaine. Further, Sample 4 released the highest amount of Lidocaine through the skin in 48 hours (FIG. 9) with significant accumulation of Lidocaine in the dermis (FIG. 10) and in the epidermis (FIG. 11). Especially when compared with release from the sample with free Lidocaine (sample 3), despite very similar loadings (˜6% for sample 3 and 5% for sample 4), sample 4 released significantly higher quantities of Lidocaine at all times. Thus, the presence of encapsulated Lidocaine together with free Lidocaine enhanced the efficiency of Lidocaine release and permeation. Importantly, combination sample 4 released more Lidocaine than the commercial sample at all times with only 25% undepleted Lidocaine versus 96% undepleted in the commercial patch at 48 hr.

It is possible that a different degradation profile of layers in the combination sample, due to synergistic effects of nanofibres with encapsulated Lidocaine, could lead to variations in permeation profiles during the transfer of Lidocaine into the skin from different types of mats.

Further Studies

Three further Franz cell studies were conducted, the results of which are outlined below. It was concluded that the patches were stable after storage at 4° C. for 4 months and that the release of Lidocaine from the combination patch started as early as 2 hours. This was approximately 2-7 fold more than that obtained from the commercial patch at all times. The patches were prepared as follows:

TABLE 9
Production of nanofibres with Lidocaine in spheres
Solutions of 10 g 12 wt % PVA with 7.5 g of paste with spheres

| | |
|---|---|
| Concentration of spheres [%] | 42.86 |
| Amount of Lidocaine [g] | 0.31 |
| Distance of electrodes [mm] | 150 |
| Voltage [kV] | −10; +25 |
| Flow rate [ml/h] | 10 |
| Unwinding speed [mm/s] | 0.1 |
| Temperature [° C.] | 21 |
| RH [%] | 33 |
| Area weight [g/m²] | 20.6 |

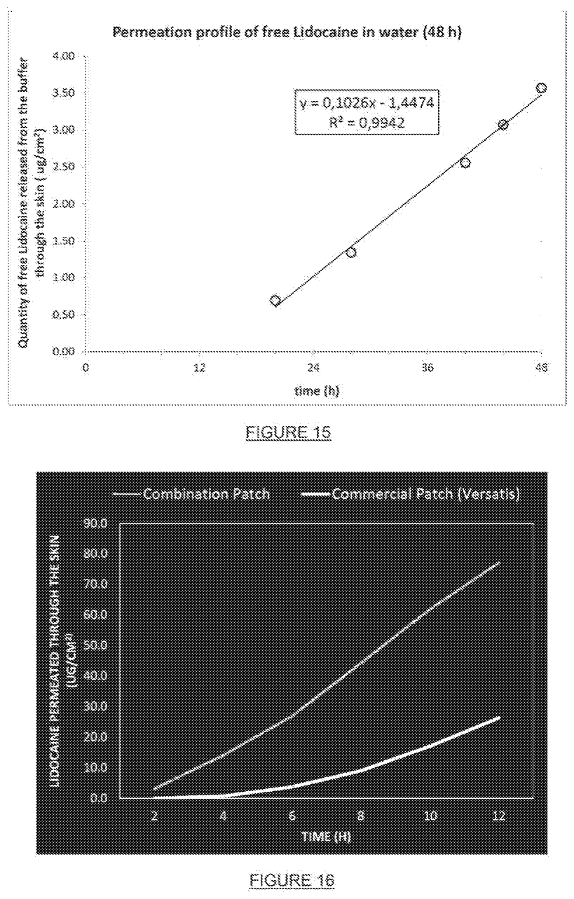

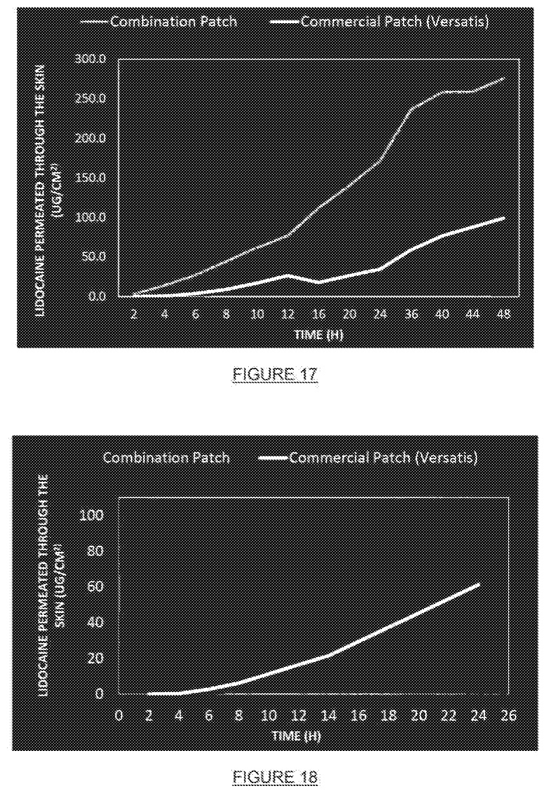

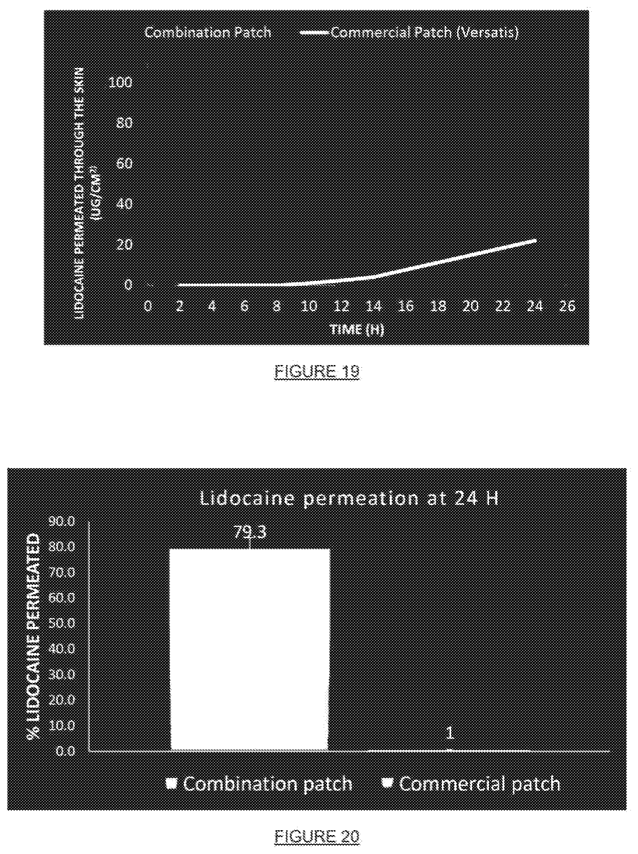

Referring to FIGS. 16-19, the performance of combination patches in three separate studies using different batches of polymer and Lidocaine particles in Franz cell experiments is illustrated. The permeation of Lidocaine from the combination patch and the commercial patch was measured through human skin (freshly obtained each time). The graph of FIG. 16 shows release of Lidocaine (through the skin) at times earlier than 12 hours. The graph of FIG. 17 shows Lidocaine released through the skin at different times for up to 48 hours. The graphs of FIGS. 18-19 depict Lidocaine released from a different batch of patches over 24 hours in two separate experiments.

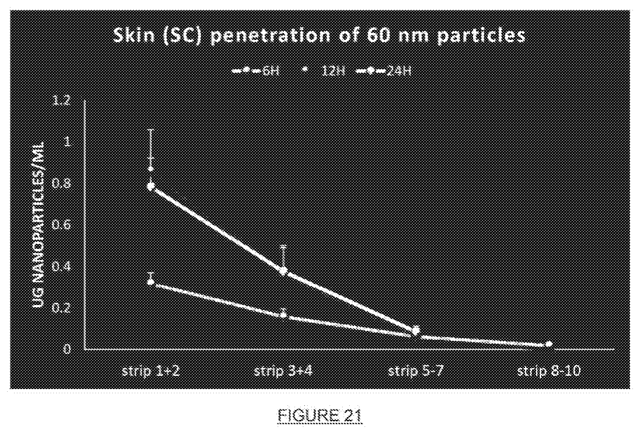

Referring to FIG. 20, a comparison of the combination patch and the commercial patch as percentage (%) of Lidocaine permeated at 24 hours through the human skin averaged over 4 separate studies is illustrated. About 80% of Lidocaine from the combination Lidocaine patch was released at 24 hours, compared with only 1% from the commercial patch.

The data from these studies reinforces the superiority of the combination Lidocaine patch over the commercial patch. The data from these studies shows that release of Lidocaine from the combination patch starts as early as 2 hours (vs. >4 hours for the commercial patch) displaying Lidocaine release at levels many folds higher than that obtained for the commercial patch at all times. Notably, this is despite the fact that the Lidocaine loading in the combination patch was 60-100 fold less than that in the commercial patch of the same dimensions.

The combination patch is consistently and significantly more efficient than the commercial patch, releasing approximately 80% of the payload at a higher release efficiency.

Penetration of Skin by Nanoparticles (50-100 nm)

Human skin was treated with combination patch containing FITC labelled silica particles (˜60 nm) in a Franz cell experiment. Stratum Corneum (SC, i.e. the outermost layer of skin consisting of keratinized dead cells) obtained from this treated skin was divided into 15 layers and levels of fluorescence was quantified in separate layers by fluorimetry over 24 hours.

Referring to FIG. 21, the fluorescence could only be detected in the top layers of stratum corneum (1-7) with near background levels in the lower layers. This implied that particles are unlikely to penetrate across the Stratum Corneum after topical application.

### CONCLUSION

Encapsulation of Lidocaine in a silica matrix was optimised using a modification of processes disclosed in International Publication No. WO 2006/133519 and Lidocaine loaded particles of appropriate size (approx. 60 nm) were produced, characterised and successfully incorporated into nanofibre nonwoven mat.

Three types of nanofibrous layers containing Lidocaine were made using the electrospinning method. Lidocaine was immobilized into nanofibrous layers directly by electrospinning of PVA solution with Lidocaine powder, a solution of PVA with particles containing Lidocaine, and a solution of PVA with a combination of Lidocaine powder and particles containing Lidocaine.

That the particles are able to release Lidocaine when incorporated into the nanofibre mat was clearly shown by USP4 evaluations. However, to compare the release kinetics of Lidocaine from different types of mats in a biologically relevant system, a Franz cell method was used. Permeation profiles of nanofibrous layers showed faster penetration through the human skin than Lidocaine in aqueous solution in the first 12 hours. This process could be caused by permeation enhancement by PVA metabolites that are transported into cells by active transport or passive diffusion. The data also clearly shows the ability of the particles with encapsulated Lidocaine to release Lidocaine through the human skin.

In general, the nanofibrous mats with Lidocaine led to improved permeation efficiencies in comparison to the commercial patch, Versatis. Despite several fold higher loadings (5000×-50000×), Versatis performed similarly to samples 2 and 3 and four times worse than sample 4 at 12 hr. This enhanced permeability of the Lidocaine from nanofibre mats could be attributed to the biodegradable nature of the fibres and due to possible permeation enhancement by PVA metabolites.

Despite 3 fold lower Lidocaine loadings, permeation profiles of nanofibrous layers with Lidocaine encapsulated in particles (sample 2) and Lidocaine powder (sample 3) were similar, indicating the permeation enhancing effect when particles containing Lidocaine are added to the fibres. However, the terminal analysis of the donor showed that sample 2 had more residual Lidocaine than sample 3, possibly indicating greater control of release of Lidocaine.

The synergistic effect of the two technologies was especially noticeable when Lidocaine was immobilised into the nanofibrous layer as a combination of ss Lidocaine containing particles and free Lidocaine (sample 4). This sample showed a very unique permeation profile.

The release rates of Lidocaine from sample 4 were superior to all types of mats including the commercial patch. This indicates the possibility of mutually beneficial effects of the two technologies. This layer showed 4 times higher release of Lidocaine through the skin than sample 2 and 3 after 12 hours. While the data showed a similar linear release of Lidocaine from sample 4 as from the commercial patch, much higher quantities were released at all times, with comparable accumulation in the deeper layers of the skin, namely the dermis.

Nanofibres containing a combination of particles with encapsulated Lidocaine and Lidocaine powder (sample 4) showed a different permeation profile compared with nanofibres containing only particles with encapsulated Lidocaine (sample 2). This phenomenon may be due to the different degradation profile of layers in the presence of the loaded particles, or some other synergistic mechanism during the transfer of Lidocaine into the skin.

This superiority of the Lidocaine nanofiber combination patches was reinforced and proven unequivocally when different batches of nanofiber patches produced using different batches of Lidocaine ceramic particles and PVA were used in three separate Franz cell experiments.

Overall, nanofibre patches performed better than the commercial patch in terms of percentage release of Lidocaine through the skin at all times. A combination of loaded particles with nanofibres appears to significantly enhance the efficiency of Lidocaine permeation. While the exact mechanism needs to be elucidated, a combination of factors may play a role in this: biodegradable nature of the fibres, permeation enhancement of PVA metabolites or mechanistic feasibility due to physical incorporation of particles in the fibres.

Unless the context requires otherwise or specifically stated to the contrary, integers, steps or elements of the invention recited herein as singular integers, steps or elements clearly encompass both singular and plural forms of the recited integers, steps or elements.

It will be appreciated that the foregoing description has been given by way of illustrative example of the invention and that all such modifications and variations thereto as would be apparent to persons of skill in the art are deemed to fall within the broad scope and ambit of the invention as herein set forth.

1.-27. (canceled)

28. A nanofibrous mat comprising:
electrospun nanofibres forming said mat; and
ceramic particles dispersed throughout said nanofibres and comprising a ceramic matrix and a dopant releasably encapsulated within said ceramic matrix,
wherein the ceramic particles are dispersed throughout the nanofibres during electrospinning of the nanofibres, whereby said dopant is protected by said ceramic matrix during said electrospinning.

29. The nanofibrous mat according to claim 28, wherein the electrospun nanofibres comprise biodegradable polymers or non-biodegredable polymers.

30. The nanofibrous mat according to claim 29, wherein the electrospun nanofibres are selected from the group consisting of cellulose acetate, collagen, elastin, gelatin, hyaluronic acid, polyacrylonitrile, polycaprolactone, polydioxanone, polyethylene oxide, polyhydroxybutyrate, poly(D-lactide), poly(D,L-lactide-co-caprolactone), poly(D,L-lactide-co-glycolide) (PLGA), polylactide, poly(L-lactide), poly(L-lactide-co-caprolactone-co-glycolide), polypropylene, polytetrafluorethylene, polyvinylpyrolidone, sodium alginate, zein and polyvinylalcohol (PVA).

31. The nanofibrous mat according to claim 30, wherein the electrospun nanofibres are formed from polyvinylalcohol (PVA), for example a 12 wt. % PVA solution.

32. The nanofibrous mat according to claim 28, wherein the dopant is poorly soluble in solvent of polymeric solution to be electrospun.

33. The nanofibrous mat according to claim 28, wherein the particles comprise solid, porous spheres, or a core with one or more layers surrounding the core.

34. The nanofibrous mat according to claim 33, wherein the particles comprise a core with one or more layers surrounding the core and wherein the dopant is located in the core, the shell or both.

35. The nanofibrous mat according to claim 28, wherein the ceramic matrix is a polymerisation and/or condensation and/or crosslinking product of a precursor material.

36. The nanofibrous mat according to claim 35, wherein the ceramic matrix comprises a hydrolysed silane, such as a hydrolysed organosilane.

37. The nanofibrous mat according to claim 35, wherein the ceramic matrix comprises an organically modified ceramic, such as an organically modified silica (organo-silica).

38. The nanofibrous mat according to claim 35, wherein the ceramic matrix comprises bound organic groups selected from ethyl, propyl, isopropyl, butyl, isobutyl, tert-butyl, pentyl, hexyl, isooctyl, decyl, dodecyl, cyclohexyl, cylcooctyl or cyclopentyl, which may be substituted or may be unsubstituted.

39. The nanofibrous mat according to claim 35, wherein the precursor material comprises an organotrialkoxysilane, and the ceramic matrix is formed in the presence of a trialkoxyaminoalkylsilane catalyst.

40. The nanofibrous mat according claim 28, wherein the dopant is selected from the group consisting of hydrophobic and hydrophilic small molecule drugs such as antibiotics (Chloremphenicol), analgesics (nonsteroidal anti-inflammatory drugs (e.g. diclofenac and ibuprofen), dibucaine, bupivacaine, capsaicin, amitriptyline, glyceryl trinitrate, opioids, menthol, pimecrolimus, and phenytoin), Scopolamine (tropane alkaloid drug) for motion sickness: proteins for therapeutic purposes, such as Steroid hormones (skin eczema or birth control, HRT, estrogen or testosterone), growth factors, cytokines, antibodies (for wound healing), vaccines (buccal patch), Nitroglycerine for Angine (sublingual patch), Vitamin B12; and fluorescent or radioactive tracers.

41. The nanofibrous mat according to claim 40, wherein the dopant is Lidocaine.

42. The nanofibrous mat according to claim 28, wherein the dopant represents between about 0.01 and 50% of the weight or the volume of the particle, or between about 0.01 and 10%, 0.01 and 1%, 0.01 and 0.5%, 0.01 and 0.1%, 0.01 and 0.05%, 0.1 and 30%, 1 and 30%, 5 and 30%, 10 and 30%, 0.1 and 10%, 0.1 and 1% or 1 and 10% of the weight or the volume of the particle.

43. The nanofibrous mat according to claim 28, wherein the ceramic particles have a diameter between about 1 nm and about 1000 nm.

44. The nanofibrous mat according to claim 28, wherein the ceramic particle diameter is <1.5 times the diameter of the fibres, more preferably smaller than the diameter of the fibres, and even more preferably ½ of the fibre diameter.

45. The nanofibrous mat according to claim 28, wherein the ceramic particles have a specific surface area of between about 2 and 400 m²/g.

46. The nanofibrous mat according to claim 28, wherein the dopant is capable of being released from the ceramic particles over a period of between about 1 minute and 2 weeks.

47. The nanofibrous mat according to claim 28, wherein the ceramic particles are in the form of a composition together with an acceptable carrier, diluent, excipient and/or adjuvant.

48. A nanofibrous mat comprising:
electrospun nanofibres forming said mat;
ceramic particles dispersed throughout said nanofibres and comprising a ceramic matrix and a dopant releasably encapsulated within said ceramic matrix; and
free dopant dispersed throughout the nanofibres.

49. A method of forming a nanofibrous mat comprising:
providing an electrospinning solution containing a precursor to electrospun nanofibres;
adding ceramic particles comprising a ceramic matrix and a dopant releasably encapsulated within said ceramic matrix to said electrospinning solution; and
electrospinning the electrospinning solution comprising said ceramic particles to form said nanofibrous mat having said ceramic particles dispersed throughout the formed electrospun nanofibers.

50. A method according to claim 49, comprising:
adding dopant in powder form to said electrospinning solution; and
electrospinning the electrospinning solution comprising the ceramic particles and dopant in powder form to form said nanofibrous mat having said ceramic particles and dopant dispersed throughout the formed electrospun nanofibers.
